(19) United States
(12) Patent Application Publication
(10) Pub. No.: US 2019/0010441 A1
(43) Pub. Date: Jan. 10, 2019

(54) OBSERVATION DEVICE, OBSERVATION METHOD, AND STORAGE MEDIUM

(71) Applicant: Olympus Corporation, Tokyo (JP)

(72) Inventor: Takeshi KINDAICHI, Tokyo (JP)

(21) Appl. No.: 16/019,299

(22) Filed: Jun. 26, 2018

(30) Foreign Application Priority Data
Jul. 4, 2017 (JP) 2017-131339

(51) Int. Cl. C12M 1/00; C12M 1/36

(52) U.S. Cl. CPC C12M 41/14; C12M 23/50; C12M 41/48

(57) ABSTRACT

An observation device that has an image sensor that forms images of a specimen, comprising: an imaging position change circuit that changes imaging position of image sensor, a temperature sensor that measures temperature within the observation device, and a controller that controls operation of the imaging position change circuit and the image sensor, wherein the controller causes execution of an operation sequence that repeatedly executes change in imaging position of the image sensor by the imaging position change circuit and imaging operation of the image sensor, stops the operation sequence if the measured temperature exceeds a given threshold, transitions to a halt sequence where a state in which change of imaging position of the image sensor and an imaging operation are prohibited continues for a given time, and repeats the operation sequence and the halt sequence.

### CROSS-REFERENCE TO RELATED APPLICATIONS

Benefit is claimed, under 35 U.S.C. § 119, to the filing date of prior Japanese Patent Application No. 2017-131339 filed on Jul. 4, 2017. This application is expressly incorporated herein by reference. The scope of the present invention is not limited to any requirements of the specific embodiments described in the application.

### BACKGROUND OF THE INVENTION

### 1. Field of the Invention

The present invention relates to an observation device and observation method that change imaging position and perform imaging using an imaging section at the changed position, and that repeatedly perform this position change and imaging operation.

### 2. Description of the Related Art

It is known to have an observation device arranged for a long period of time within a constant temperature oven or incubator that keeps an environment, such as temperature, constant, and observe a sample such as cells within a culture vessel using an imaging section. This observation device normally has a sealed structure in order to maintain electrical performance even within a constant temperature oven or an incubator or the like. When forming an image of cells or the like using an imaging section, since electronic components etc. of the imaging section generate heat, temperature around the observation device within the temperature controlled oven, specifically the temperature of a sample such as cells within a culture vessel, will change.

Also, with a digital camera or the like, if the time taken to acquire a live view image becomes long internal devices such as the image sensor generates heat, and not only will the internal temperature within the camera rise, but image quality of a still image of a paused live view image will be degraded. A digital camera has therefore been proposed that prevents degradation in image quality of a still image taken immediately after stopping live view display by changing display processing of a live view image based on temperature of an image sensor at the point in time that live view display is instructed (refer to Japanese patent laid-open No. 2009-033508 (hereafter called “patent publication 1”)).

In an observation device for cells etc. that have been placed inside an incubator, an imaging section of the observation device is placed within the incubator that has been sealed, while the camera disclosed in patent publication 1 is within a space that is open, and so the circumstances of the two devices are different. Specifically, in patent publication 1 no consideration has been given to rise in temperature affecting things outside the camera. Effects also arise such as change in temperature of a sample that has been placed adjacent to the observation device or on the observation device due to change in temperature inside an incubator or the like. However, with temperature control such as is disclosed in patent publication 1, it is not possible to avoid the effects of temperature change on cells that constitute a sample, and cell cultivation etc. is disrupted.

### SUMMARY OF THE INVENTION

An object of the present invention is to provide an observation device and an observation method that are capable of acquiring images using an imaging section while keeping temperature within a given range, taking into consideration the effect that rise in temperature of the observation device has on a sample.

A observation device of a first aspect of the present invention has an image sensor that forms images of a specimen, this observation device comprising: an imaging position change circuit that changes imaging position of the image sensor, a temperature sensor that measure temperature of the inside of the observation device and outputs results of this measurement as a measured temperature, and a controller that controls operation of the imaging position change circuit and the image sensor, wherein, the controller causes execution of an operating sequence that repeatedly executes change of imaging position of the image sensor by the imaging position change circuit and an imaging operation of the image sensor, causes transfer to a halt sequence where the operating sequence is stopped if the measured temperature exceeds a given threshold value, and where a state in which change of imaging position of the image sensor and an imaging operation are prohibited continues for a given time, and repeatedly executes the operating sequence and the halt sequence.

An observation method of a second aspect of the present invention is an observation method for an observation device having an image sensor that forms images of a specimen, this observation method comprising: changing imaging position of the image sensor, and measuring temperature inside the observation device and outputting this measurement result as measured temperature, and when controlling imaging position of the image sensor and operation of the image sensor, causing execution of an operating sequence that repeatedly executes change of imaging position of the image sensor and an imaging operation of the image sensor, and transferring to a halt sequence, where the operating sequence is stopped if the measured temperature exceeds a given threshold value, and where a state in which change of imaging position of the image sensor and an imaging operation are prohibited continues for a given time.

A storage medium of a third aspect of the present invention is a storage medium provided within a computer of an observation device that has an image sensor for forming images of a specimen, the storage medium storing a program for executing an observation method, the observation method comprising: changing imaging position of the image sensor, measuring temperature inside the observation device and outputting this measurement result as measured temperature, and when controlling imaging position of the image sensor and operation of the image sensor, causing execution of an operating sequence that repeatedly executes change of imaging position of the image sensor and an imaging operation of the image sensor, and transferring to a halt sequence, where the operating sequence is stopped if the measured temperature exceeds a given threshold value, and where a state in which change of imaging position of the image sensor and an imaging operation are prohibited continues for a given time.

### BRIEF DESCRIPTION OF THE DRAWINGS

FIG. 1 is a block diagram showing overall structure of a cell observation system of one embodiment of the present invention.

FIG. 2 is an external perspective view showing a cell observation device, vessel, and operating sections of one embodiment of the present invention.

FIG. 3 is a block diagram mainly showing the electrical structure of a cell observation device of one embodiment of the present invention.

FIG. 4A to FIG. 4C are external perspective views showing one example of a vessel for holding cells, in a cell observation system of one embodiment of the present invention.

FIG. 5 is a drawing showing operational overview of cell culture observation of the cell observation system of the one embodiment of the present invention.

FIG. 6 is a drawing showing details of a batch operation, in the cell observation system of the one embodiment of the present invention.

FIG. 7 is a graph showing change in internal temperature at the time of operation on an imaging section and in a cooling period, in the cell observation system of one embodiment of the present invention.

FIG. 8 is a flowchart showing operation of an imaging section within the cell observation device of one embodiment of the present invention.

FIG. 9 is a flowchart showing operation of an information terminal of one embodiment of the present invention.

FIG. 10 is a flowchart showing preproject operation in the cell observation device of one embodiment of the present invention.

FIG. 11 is a flowchart showing operation of batch processing in the cell observation device of one embodiment of the present invention.

### DETAILED DESCRIPTION OF THE PREFERRED EMBODIMENTS

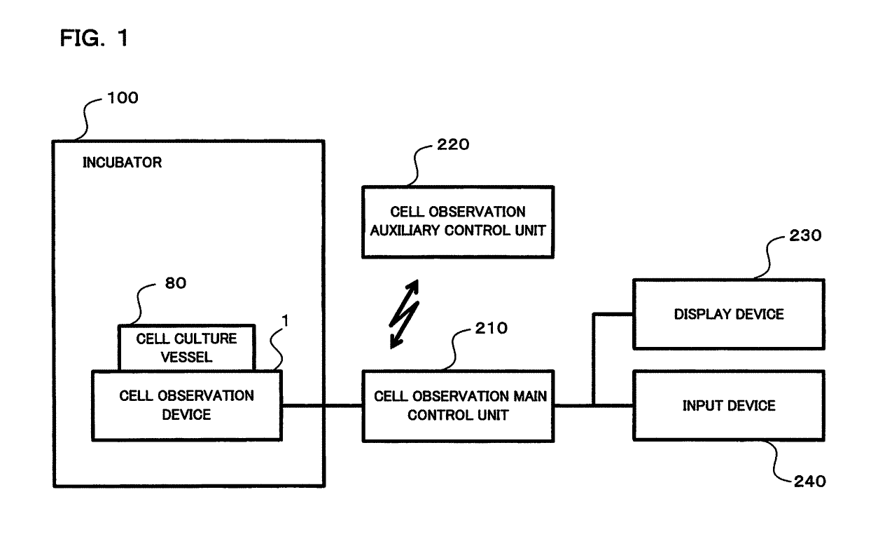

One embodiment of the present invention applied to a cell observation system will be described in the following using the drawings. FIG. 1 is a block diagram showing overall structure of a cell observation system. This cell observation system comprises an incubator 100, a cell culture vessel 80, a cell observation device 1, a cell observation main control unit 210, a cell observation auxiliary control unit 220, a display device 230 and an input device 240.

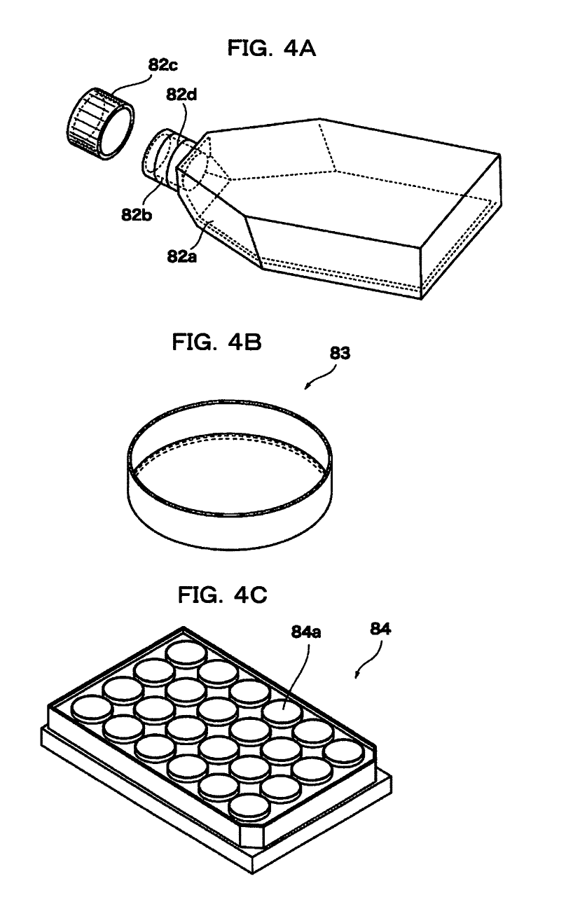

The incubator 100 has a sealed structure, with the inside being kept at a constant temperature (for example, 37.0° C.) by being subjected to temperature adjustment. The inside of the incubator 100 may also have humidity, oxygen concentration and carbon dioxide concentration (for example, 5.0%) kept constant. The cell culture vessel 80 is a bottle or petri dish for cultivation of cells, and has various sizes shapes and materials. Temperature etc. is kept constant within the incubator 100, and under this environment cells are cultivated within the cell culture vessel 80. The shape etc. of the cell culture vessel 80 will be described later using FIG. 4.

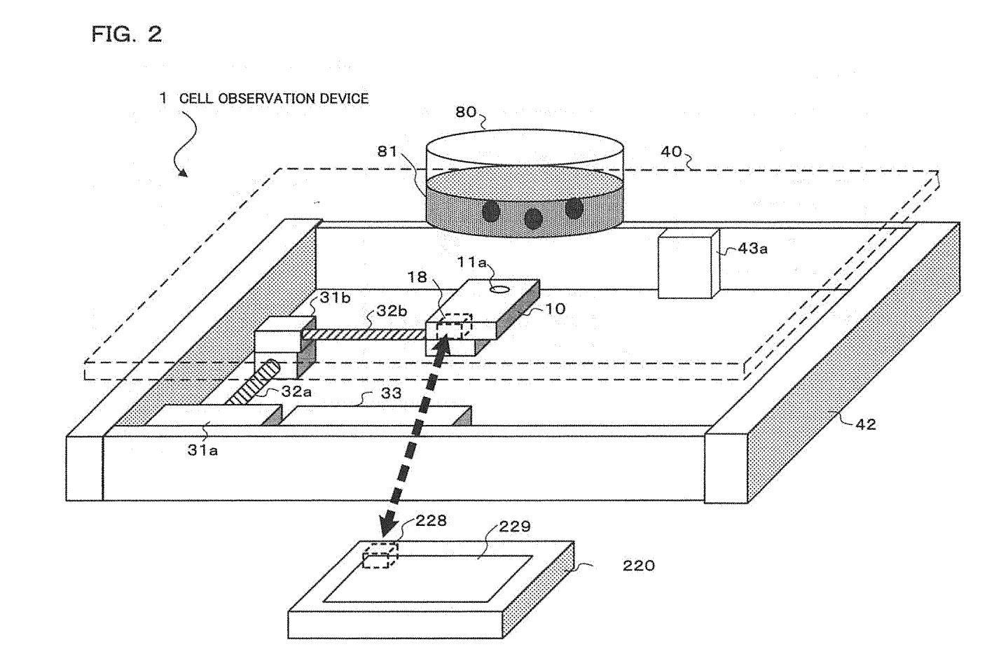

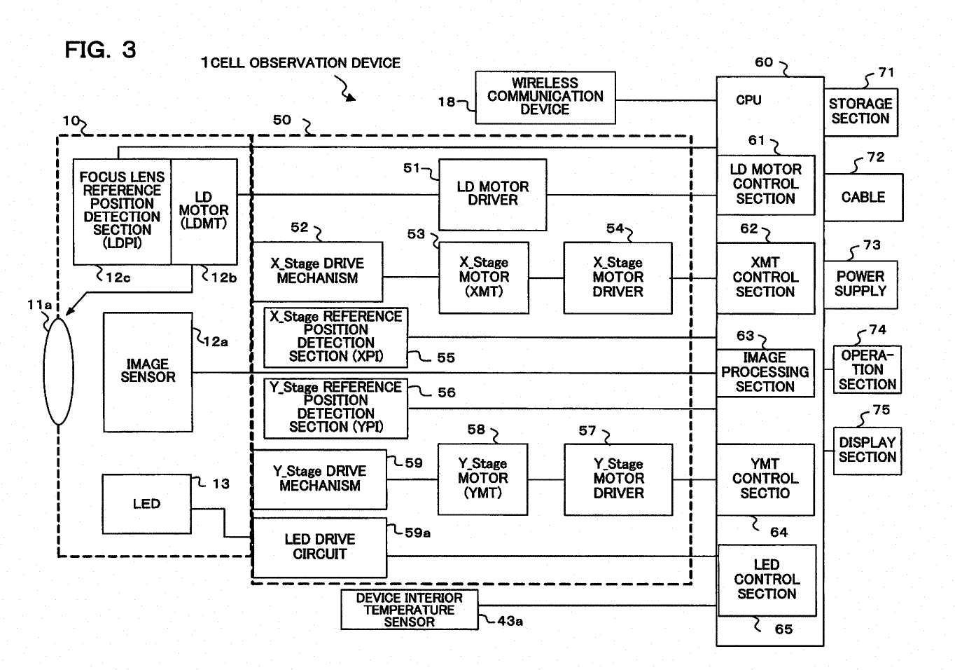

The cell observation device 1 is arranged within the incubator 100, and has a camera section (imaging section) 10 which will be described later. Cells within the cell culture vessel 80 are observed using the camera section 10. Also, the camera section 10 can be moved in x axis and y axis directions, as will be described later, and position of the camera section 10 is automatically changed in line with a pattern that is set in advance. The camera section 10 acquires images of cells within the cell culture vessel 80 at the changed position. It should be noted that not only is the camera section 10 moved automatically in line with a predetermined pattern, it is also possible to move the camera section 10 to an arbitrary position as a result of manual operation by the user, and acquire images of cells. Detailed appearance and electrical structure of this cell observation device 1 will be described later using FIG. 2 and FIG. 3.

The cell observation main control unit 210 is a personal computer (PC) or the like. This cell observation main control unit 210 can communicate with the cell observation device 1 by means of wired communication or wireless communication, and performs overall control of the cell observation device 1. It should be noted that besides a PC, the cell observation main control unit 210 may be a control device such as a server, and the cell observation device 1 may connect to this control device by means of an intranet or the like.

The display device 230 and the input device 240 are connected to the cell observation main control unit 210. This connection may be a wired connection, and may be a wireless connection. The display device 230 has a display section such as a monitor, and displays live images and/or playback images. Live images are images at the time of observation that are acquired by the imaging section 10 of the cell observation device 1. Also, playback images are images that have been played back after reading out stored images by the cell observation main control unit 210. Also, menu screens etc. and also screens for various settings such as mode setting are displayed on this display section.

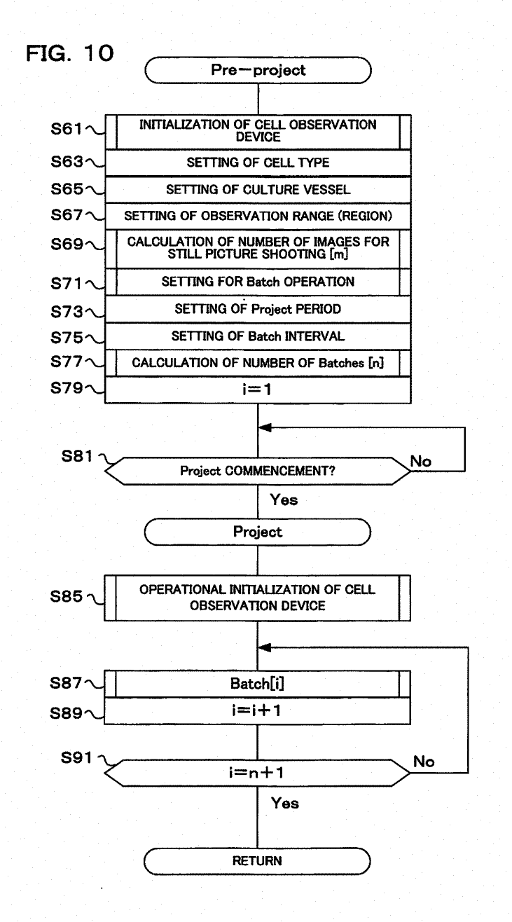

The input device 240 has an input interface such as a keyboard. Using the input device 240, the user sets various modes and inputs information etc. such for the cell culture vessel 80. The input device 240 or the display section 229 of the cell observation auxiliary control unit 220 (refer to FIG. 2) functions as an input section (US: interface) that inputs information of vessels in which specimens are placed (refer, for example, to S65 in FIG. 10, which will be described later). This input section inputs information relating to vessels of a plurality of different shapes (refer, for example, to FIG. 4A to FIG. 4C, and S65 in FIG. 10, which will be described later).

The cell observation auxiliary control unit 220 is an information terminal that has portability, and may be a dedicated unit. Also, a smartphone or tablet PC etc. may be used as a cell observation auxiliary control unit. Also, the cell observation auxiliary control unit 220 communicates with the cell observation main control unit 210 or the cell observation device 1 by means of wireless communication, and can control the cell observation system. As wireless communication, as well as wireless communication such as WiFi etc. there may also be infrared communication. If the display section is provided in the cell observation auxiliary control unit 220, it is possible to display live images that have been acquired using the cell observation device 1, and playback display of stored images is possible.

At least one of the cell observation main control unit 210 and the cell observation auxiliary control unit 220 functions as an external control unit that can communicate with the observation device. The input section (for example, the input device 240) is input with information relating to vessels of a plurality of different shapes by receiving instruction from the external control unit (refer, for example, to S65 in FIG. 10).

This type of cell observation system of this embodiment has the cell culture vessel 80 arranged within the incubator 100, and acquires image data by imaging cells that are cultivated within this cell culture vessel 80 using the imaging section 10 within the cell observation device 1. This image data that has been acquired is output to the cell observation main control unit 210 and/or the cell observation auxiliary control unit 220, and the image data that has been output is displayed on the display device 230. This means that at the time of observing cells by the user, it is possible for the cell culture vessel 80 to cultivate cells in an environment such as constant temperature, without carrying out from the incubator.

Next, the main structure of the cell observation device 1 will be described using the external perspective view shown in FIG. 2. FIG. 2 shows the cell observation device 1, cell culture vessel 80, and cell observation auxiliary control unit 220 within the cell observation system. As was described previously, within this cell observation system the cell observation device 1 and the cell culture vessel 80 are arranged within the incubator 100, and the cell observation auxiliary control unit 220 is arranged outside of the incubator 100. It should be noted that this may also apply to the cell observation main control unit 210 instead of the cell observation auxiliary control unit 220.

It is possible to mount the cell culture vessel 80 on a transparent top board 40 of the cell observation device 1, and cells (specimen 81) are cultivated within the cell culture vessel 80. The imaging section 10 forms images of the specimen 81 through the transparent top board 40, and can acquire image data of the formed images. This means that cells (the specimen 81) are cultivated directly within the incubator 100 with an environment maintained, and it is possible to perform measurement and observation of the specimen 81 etc. remotely in the cell observation auxiliary control unit 220 and/or the cell observation main control unit 210 etc. that are outside the incubator 100. Since the cell observation device 1 is arranged within the incubator 100, energy release by the cell observation device 1 is significant and there is an increase in temperature within the incubator 100. There is also cultivation in an environment of high temperature and high humidity. It is therefore preferable for the cell observation device 1 to be designed with high energy saving capability and reliability.

The cell observation device 1 has a camera section 10, Y actuator 31a, X actuator 31b, Y feed screw 32a, X feed screw 32b, movement control section 33, transparent top board 40, outer housing 42 and device interior temperature sensor 43a. The camera section 10 has a lens 11a, with an image that has been formed by the lens 11a being subjected to photoelectric conversion by an image sensor 12a (refer to FIG. 3) to acquire image data. Also, a wireless communication device 18 is arranged within the cell observation device 1, and wireless communication is possible with a communication section 228 within the cell observation auxiliary control unit 220 that is arranged externally to the cell observation device 1. The detailed structure of the camera section 10 will be described later using FIG. 3.

The camera section 10 is held on an X feed screw 32b, and is capable of moving in the X axis direction by rotation of the X feed screw 32b. The X feed screw 32b is driven to rotate by the X actuator 31b. The X actuator 31b is held on the Y feed screw 32a, and is capable of movement in the Y axis direction by rotation of the Y feed screw 32a. The Y feed screw 32a is driven to rotate by the Y actuator 31a.

The movement control section 33 has a drive control circuit, and performs drive control for the Y actuator 31a and the X actuator 31b, and moves the camera section 10 in the X axis and Y axis directions in accordance with a procedure that has been preprogrammed. Also, the user can cause the camera section 10 to move to a specified position. In this case, the user issues an instruction by manual operation using the cell observation auxiliary control unit 220, and the movement control section 33 causes the camera section 10 to move in accordance with this instruction. The movement control section 33, Y actuator 31a, X actuator 31b etc. function as an imaging position change section that changes imaging position of the imaging section. Also, the drive control circuit that controls drive of the Y actuator and the X actuator 31b functions as an imaging position change circuit that changes imaging position of the image sensor.

It should be noted that a built in power supply battery 73 is provided within the cell observation device 1, as will be described later. The movement control section 33, Y actuator 31a, X actuator 31b and camera section 10 are supplied with power from the power supply battery 73. Also, communication lines for bidirectional communication of control signals are provided between each of the sections within the cell observation device 1. With this embodiment it is assumed that a power supply battery is used as the power supply, but this is not limiting, and supply of power may also be implemented using an AC power supply. It is also assumed that control signals between each of the sections are interchanged by means of wired communication, but it is also possible to use wireless communication.

The above described camera section 10, Y actuator 31a, X actuator 31b, Y feed screw 32a, X feed screw 32b, and movement control section 33 are arranged inside a housing that is made up of a top plate 40 and an outer housing 42. The top plate 40 and outer housing 42 constitute an encapsulating structure such that moisture does not infiltrate into the inside of the housing from outside. As a result the inside of the housing constituted by the top plate 40 and the outer housing 42 are not subjected to high humidity, even if the inside of the incubator 100 is high humidity.

A device interior temperature sensor 43a is arranged as a built in sensor inside the housing that is constituted by the top plate 40 and outer housing 42. As the built in sensor, in this embodiment there is a temperature sensor for detecting temperature inside the cell observation device, but this is not limiting and it is also possible to have a voltage sensor for detecting voltage, a humidity sensor for detecting humidity, etc. Besides the temperature sensor, there may be only one of a voltage sensor and a humidity sensor, and a sensor for detecting another parameter may also be arranged. Also, arrangement position of the sensors does not need to be at a single location, and the sensors may be arranged suitably dispersed.

It should be noted that external sensors may also be arranged outside the casing that is made up of the top plate 40 and the outer housing 42. As the external sensors there are a temperature sensor for detecting temperature outside the cell observation device 1, a voltage sensor for detecting voltage, a humidity sensor for detecting humidity, an oxygen concentration sensor for detecting oxygen concentration, a nitrogen concentration sensor for detecting nitrogen concentration, a carbon dioxide concentration sensor for detecting carbon dioxide concentration, etc. There may be one or a plurality of any of these sensors, and sensors for detecting other items may also be arranged. Also, arrangement position of the sensors does not need to be at a single location, and the sensors may be arranged suitably dispersed.

It is possible to mount the cell culture vessel 80 on an upper side of the transparent top board 40. The inside of the cell culture vessel 80 is filled with a culture medium, and it is possible to cultivate a specimen 81 (cells). The image sensor 12a of the camera section 10 forms images of the culture medium inside the cell culture vessel 80 through the transparent top board 40, and it is possible to observe images. In order to form images of the cells within the cell culture vessel 80 by the camera section 10, the bottom surface of the cell culture vessel 80 (side in contact with the top plate 40) is preferably transparent.

Also, the cell observation device 1 can count cells etc. of the specimen 81 by analyzing images that have been taken. Specifically, the camera section 10 acquires images of the specimen 81 within the cell culture vessel 80 while being moved by the X actuator 31b and the Y actuator 31a, and it is possible to count a number of cells etc. based on the images that have been acquired.

The cell observation auxiliary control unit 220 has a communication section 228, and can communicate with the wireless communication device 18 within the cell observation device 1. This means that it is possible for the cell observation auxiliary control unit 220 to carry out communication with the camera section 10 at a position that is remote from the cell observation device 1, and it is possible to move the camera section 10 and to receive image data that has been acquired by the camera section 10. It should be noted that the cell observation auxiliary control unit 220 may be a dedicated unit, but an information terminal device such as a smartphone may also double as an operation section.

The cell observation auxiliary control unit 220 also has a display section 229. The display section 229 performs displays of icons for various modes of the cell observation auxiliary control unit 220 and for various settings etc. If a touch panel is provided, it is possible to carry out various inputs using a touch operation. The display section 229 also displays images that have been acquired and transmitted by the camera section 10. It should be noted that if image display is not necessary in the cell observation auxiliary control unit 220 the display section 229 may be omitted.

Next, the electrical structure of the cell observation device 1 of this embodiment will mainly be described using FIG. 3. The cell observation device 1 has the camera section 10, X/Y stage section 50, CPU (Central Processing Unit) 60, device interior temperature sensor 43a, and other peripheral circuits etc.

There is a photographing lens that includes a focus lens 11a within the camera section 10. The photographing lens that includes this focus lens 11a is a prime lens or a zoom lens, and forms images of the specimen 81, such as cells etc., on the image sensor 12a. While FIG. 3 does not show an aperture and mechanical shutter on the optical axis of the focus lens 11a, these components may be provided, or they may be omitted.

The image sensor 12a is an image sensor such as a CCD (Charge Coupled Device) or CMOS (Complementary Metal-Oxide-Semiconductor) image sensor. The image sensor 12a generates an image signal by subjecting an image that has been formed by the focus lens 11a to photoelectric conversion. This image signal is subjected to processing such as A/D conversion in an imaging circuit to generate image data, and this image data is output to an image processing section 63. The image sensor 12a functions as an image sensor that forms images of the specimen. The image sensor is capable of imaging corresponding to still images, imaging corresponding to live view, and imaging corresponding to movies.

The focus lens 11a is moved in the optical axis direction of the focus lens 11a by a lens drive (LD) motor (LDM) 12b. The LD motor 12b is a stepping motor in this embodiment, but maybe another type of motor.

A focus lens reference position detection section (LDPI) 12c has a photo interrupter or the like, and outputs a reference position signal to the CPU 60 when the focus lens 11a reaches a reference position. The point in time when this reference position signal has been output is made a reference time point, and it is possible to detect position of the focus lens 11a based on a number of pulses that have been applied to the stepping motor. Based on this position detection an LD motor control section 61 can move the focus lens 11a to a focus position that is made a target position. It should be noted that in the case of a mode to other than a stepping motor, a sensor may be provided to detect relative or absolute position of the focus lens 11a.

An LED (Light Emitting Diode) 13 is a light source for illuminating cells (specimen 81) within the cell culture vessel 80. Emission wavelength of the LED is preferably red light that does not damage the cells (specimen 81), and more specifically may be set to red light having a wavelength of 660 nm. It should be noted that a light source other than an NED may be used as a light source of a lighting unit.

The X/Y stage section 50 is a mechanism for moving position of the camera section 10 in the X direction and in the Y direction. The Y actuator 31a, X actuator 31b, movement control section 33 etc. of FIG. 1 are equivalent to the X/Y stage section 50 of FIG. 3.

An LD motor driver 51 is a drive circuit for the LD motor 12b, and outputs drive pulses for the LD motor 12b in accordance with a control signal from the LD motor control section 61 within the CPU 60. The above-described LD motor 12b and the LD motor driver 51, and the LD motor control section 61 which will be described later, function as a focus section that controls focusing of the imaging section. The above described LD motor 12b and the LD motor driver 51, and the LD motor control section 61 which will be described later, function as a focus control circuit that controls focusing of the image sensor. The focus control circuit is capable of adjusting focus of the focus lens 11a such that contrast becomes a peak, based on image information that has been formed by the image sensor.

An X stage drive mechanism 52 is a mechanism for driving the camera section 10 in the X axis direction, and is equivalent to the X feed screw 32b in FIG. 2. It should be noted that the X stage drive mechanism 52, besides being a feed screw mechanism, may be a mechanism for causing the camera section 10 to move spatially in the X axis direction, such as a gear mechanism or a belt mechanism or the like.

An X stage motor (XMT) 53 is a motor for causing rotation of the X feed screw 32b, and in this embodiment a stepping motor is adopted. The X stage motor 53 is equivalent to the X actuator 31b in FIG. 2. The X stage motor 53 is pulse driven by an X stage stepping motor driver 54. The X stage stepping motor driver 54 performs drive control of the X stage motor 53 in accordance with a control signal from an XMT control section 62 within the CPU 60.

An X stage reference position detection section 55 has a detection sensor such as a PI (photo interrupter) or PR (photo reflector), and outputs a reference signal to the CPU 60 when the camera section 10 has reached a reference position in the X axis direction. The XMT control section 62 can move the camera section 10 to a target position in the X axis direction by applying a given number of pulses to the X stage motor 53 from the point in time when this reference position has been reached.

A Y stage drive mechanism 59 is a mechanism for driving the camera section 10 in the Y axis direction, and is equivalent to the Y feed screw 32a in FIG. 2. It should be noted that the Y stage drive mechanism 59, besides being a feed screw mechanism, may be a mechanism for causing the camera section 10 to move spatially in the Y axis direction, such as a gear mechanism or a belt mechanism or the like.

A Y stage motor (YMT) 58 is a motor for causing rotation of the Y feed screw 32a, and in this embodiment a stepping motor is adopted. The Y stage motor 58 is equivalent to the Y actuator 32a in FIG. 2. The Y stage motor 58 is pulse driven by a Y stage stepping motor driver 57. The Y stage stepping motor driver 57 performs drive control of the Y stage motor 58 in accordance with a control signal from a YMT control section 64 within the CPU 60.

A Y stage reference position detection section 56 has a detection sensor such as a PI (photo interrupter) or PR (photo reflector), and outputs a reference signal to the CPU 60 when the camera section 10 has reached a reference position in the Y axis direction. The YMT control section 64 can move the camera section 10 to a target position in the Y axis direction by applying a given number of pulses to the Y stage motor 58 from the point in time when this reference position has been reached.

The above described X stage stepping motor driver 54, X stage motor 53, X stage drive mechanism 52, X stage reference position detection section 55, Y stage stepping motor driver 57, Y stage motor 58, Y stage drive mechanism 59 and Y stage reference position detection section 56 function as an imaging position change section that changes imaging position of the imaging section. Also, the above described X stage stepping motor driver 54, X stage motor 53, X stage reference position detection section 55, Y stage stepping motor driver 57, Y stage motor 58, and Y stage reference position detection section 56 function as an imaging position change circuit that changes imaging position of the image sensor.

An LED drive circuit 59a performs lighting control for the LED 13 in accordance with a control signal from an LED control section 65 within the CPU 60. If illumination is performed using the LED 13, the LED etc. produces heat, temperature inside and outside the cell observation device 1 rises, and it is not possible to keep the temperature of the cell observation device 1 and the incubator 100 constant. Illumination time is therefore controlled, as will be described later. It is also possible to control light emission brightness.

The CPU 60 performs control for each section within the cell observation device 1 in accordance with a program that is stored in a storage section 71. Within the CPU 60 there are the lens drive (LD) motor control section 61, X motor (XMT) control section 62, image processing section 63, Y motor (YMT) control section 64, and LED control section 65. Each of these sections is realized in a software manner using a program, but some functions of each of the control section 61, 62, 64 and 65 and/or the image processing section 63 etc. may be realized using hardware circuits etc.

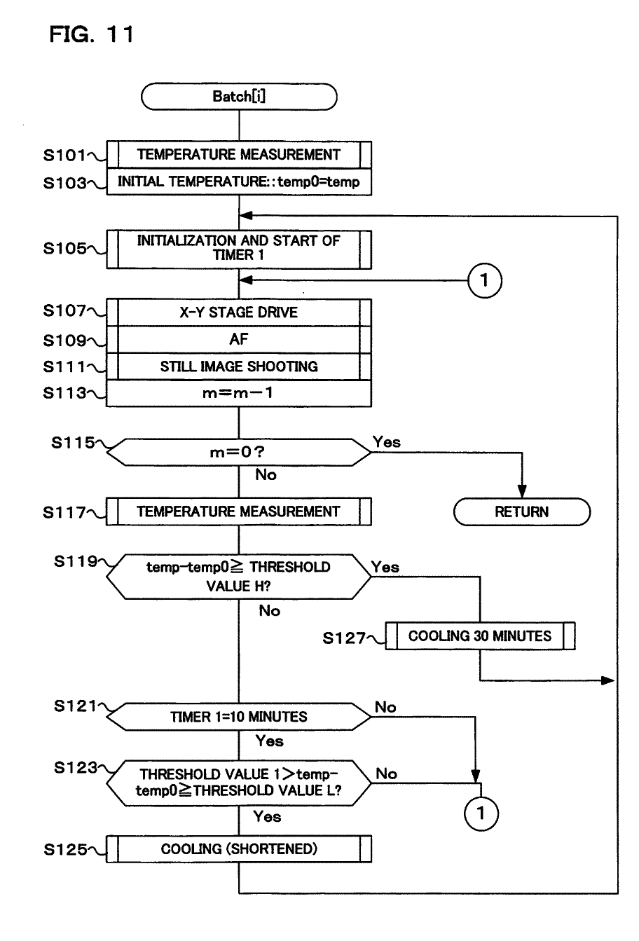

The CPU 60 functions as a controller that controls operation of the imaging position change circuit and the image sensor. This controller executes an operation sequence that repeatedly executes change in imaging position of the image sensor by the imaging position change circuit and an imaging operation of the image sensor (refer, for example, to S107 to S111 in FIG. 11), and, if a temperature that has been measured exceeds a given threshold value (refer, for example, to S119 Yes in FIG. 11), transitions to a halt sequence where the operation sequence is stopped and a state where change of imaging position of the imaging section and the imaging operation are not executed continues for a given time (refer, for example, to S127 in FIG. 11). The controller also changes the threshold value or given time based on input interface information.

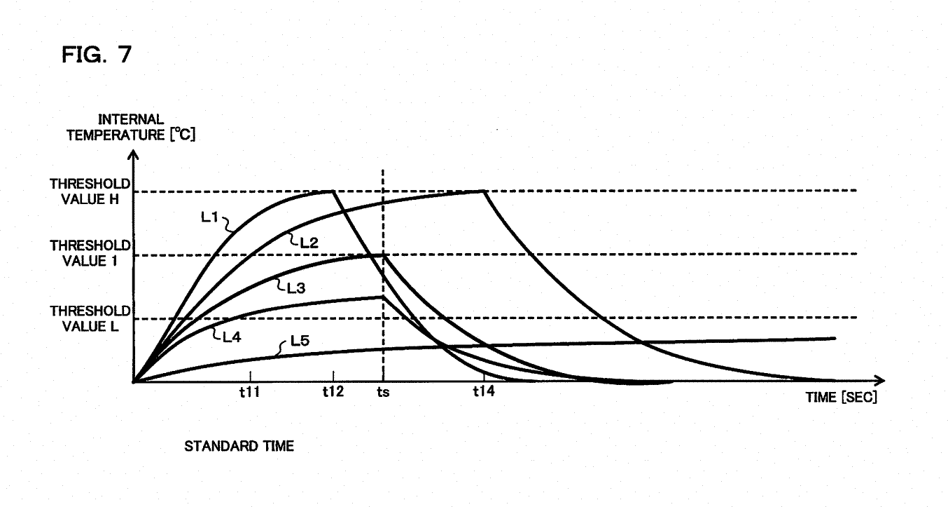

In the event that measured temperature does not exceed a first threshold value (for example, threshold value H in FIG. 7) until a second given time (for example, standard time 10 minutes in FIG. 7) has elapsed from commencement of the operation sequence, the controller continues the operation sequence even after the second given time has elapsed (for example, temperature curve L2 in FIG. 7, and S119 No and S123 No in FIG. 11), while if measured temperature exceeds the first threshold value in the period from commencement of the operation sequence until the second given time has elapsed the controller transitions to the halt sequence where the operation sequence is stopped, and continues the halt sequence for a first given time (for example, cooling period 30 minutes) (refer, for example, to temperature change curve L1 in FIG. 7, and S119 Yes and S127 in FIG. 11). It should be noted that in FIG. 7 and FIG. 11, which will be described later, three threshold values are provided. However, control may focus on the first threshold value, as described above.

In the event that measured temperature exceeds a second threshold value (for example, threshold value L in FIG. 7) in a period until the second given time (for example, standard time 10 minutes in FIG. 7) has elapsed from commencement of the operation sequence, and a third threshold value (for example threshold value 1 in FIG. 7) that is larger than the second threshold value and smaller than the first threshold value (for example threshold value H in FIG. 7) is not exceeded, the controller transitions to the halt sequence where the operation sequence is stopped, and continues the halt sequence for a third given time that is smaller than the first given time (refer, for example, to temperature change curve L4 in FIG. 7, and S123 Yes and S125 in FIG. 11), while if measured temperature exceeds the third threshold value in a period until the second given time has elapsed from commencement of the operation sequence, the controller transitions to the halt sequence where the operation sequence is stopped, and continues the halt sequence for the given time (for example, cooling period 30 minutes) (refer, for example, to temperature change curve L3 in FIG. 7).

Observation is possible by utilizing a plurality of vessels of different shapes in which samples are arranged (refer to FIG. 4), and the controller changes the first threshold value (refer, for example, to threshold value H in FIG. 7) and the second threshold value (refer, for example, to threshold value L in FIG. 7) depending on information specifying a vessel that has been adopted from among the plurality of vessels of different shapes. Also, the third threshold value (refer, for example, to threshold value 1 in FIG. 7) is changed in accordance with information specifying a vessel that is used from among the plurality of vessels of different shapes. The controller specifies a vessel that a specimen is placed in from among the plurality of vessels of different shape based on information that is input using an interface (refer, for example, to S65 in FIG. 10).

It should be noted that the controller may also repeatedly execute change of imaging position of the image sensor using the imaging position change circuit and the imaging operation of the image sensor, and specify a vessel in which a sample is placed from among a plurality of vessels of different shape based on image data that is generated by the image sensor. Specifically, during batch processing or the like, since images of the cell culture vessel 80 are acquired, a vessel may be specified based on the images that have been acquired.

During execution of the operation sequence the controller changes imaging position of the image sensor using the imaging position change circuit, performs focusing by controlling relative focus of the image sensor using the focus control circuit (refer, for example, to S109 in FIG. 11), and executes an imaging operation using the image sensor, but during the halt sequence the focus control circuit is not operated. In steps S125 and S127 that will be described later, focus control is not performed.

The controller executes the operation sequence to repeatedly execute change of imaging position by the imaging position change circuit and an imaging operation of the image sensor, and if a measured temperature exceeds a first threshold value (refer, for example, to threshold value H in FIG. 7) the operation sequence is stopped and there is a transition to a halt sequence (refer, for example, to S127 in FIG. 11) where a state in which the change of imaging position of the image sensor and the imaging operation are not performed continues for a first given time, and when a second given time (for example the 10 minutes of S121 in FIG. 11) has elapsed from commencement of the operation sequence if measured temperature exceeds a second threshold value (for example, threshold value L in FIG. 7) that is smaller than the first threshold value, and does not exceed the first threshold value, the operation sequence is stopped and there is a transition to the halt sequence, and the first given time is switched to a third given time that is smaller than the first given time (for example, S125 in FIG. 11) (refer, for example, to temperature change curves L1 and L4 in FIG. 7).

The LD motor control section 61 has a motor drive circuit, and focusing of the focus lens 11a is controlled by performing drive control of the lens drive (LD) motor 12b (refer to S109 in FIG. 11 which will be described later). With this embodiment, focus of the focus lens 11a is adjusted by moving the focus lens 11a in the optical axis direction. However, focus of the focus lens 11a may also be adjusted by moving the image sensor 12a. That is, relative focus control may be performed by adjusting the distance between the focus lens 11a and the image sensor 12a. The XMT control section 62 performs positional adjustment of the camera section 10 in the X axis direction by performing drive control of the X stage motor 53. The YMT control section 64 performs positional adjustment of the camera section 10 in the Y axis direction by performing drive control of the Y stage motor 58 (refer to S107 in FIG. 11 which will be described later).

The image processing section 63 is input with an image signal from the image sensor 12a, and has image processing circuits that subject this image signal to various image processing. The image processing section 63 processes the image signal from the image sensor 12a and performs image display on the display section 75. Also, the image signal that has been processed by the image processing section 63 is transmitted to an external display section (for example, the display section 229 of the cell observation auxiliary control unit 220) by means of a communication cable within a cable 72, and the external display section can display images of the specimen 81 etc. The image processing section 63 may also perform image analysis such as counting a number of specified cells within the specimen 81. Further, the image processing section 63 calculates an AF (Auto Focus) evaluation value based on image data. The AF evaluation value is a value that represents focused state of the focus lens 11a.

The LED control section 65 controls light emission of the LED 13 using the LED drive circuit 59a.

The storage section 71 has electrically rewritable volatile memory and/or electrically rewritable non-volatile memory. The storage section 71 stores various adjustment values of the cell observation device 1, as well as the previously described program. Also, a user presets a movement path (movement pattern) of the camera section 10 that is performed at the time of cell observation, and the storage section 71 may also store this movement pattern. That is, a designated position for performing still picture shooting, and shooting condition at this designated position, are stored in the storage section 71 as a set.

The cable 72 is a communication cable for when connecting between the cell observation device 1 and the cell observation auxiliary control section 220 in a wired manner. Images that have been acquired by the image sensor 12a are transmitted externally by means of this communication cable. In the case of wireless communication, this communication cable can be omitted. The cable 72 is also a power supply cable in the case of supplying power to the cell observation device 1 from outside. In the event that a power supply battery is built into the cell observation device 1, the cable may be omitted. A power supply section 73 receives supply of power from outside by means of the cable 72 or using the built-in battery, and converts to a power supply voltage used by the cell observation device 1.

The operation section 74 has a switch or the like for turning a power supply on or off and a switch or the like for turning communication, such as wireless communication with the cell observation auxiliary control section 220, on or off. Besides this, the operation section 74 may also include switches, a dial or a touch panel etc. for performing some operations using the cell observation auxiliary control section 220. The display section 75 has a display, and displays images of the specimen 81 etc., that have been processed by the image processing section 63. It should be noted that the display section 75 may be omitted and images displayed only on an external unit.

The previously described device interior temperature sensor 43a detects temperature inside the housing that is made up of the top plate 40 and the outer housing 42, and outputs the detected temperature to the CPU 60. The device interior temperature sensor 43a functions as a temperature sensor that measures temperature inside the observation device, and outputs the result of this measurement as measured temperature. Also, the device interior temperature sensor 43a is arranged only at a single location in FIG. 2. However, a plurality of sensors may be arranged at different positions within the housing. In this case, a controller may estimate the temperature of the specimen 81 based on output of the plurality of temperature sensors, and decide on a temperature to be measured.

The cell observation device 1 of this embodiment has the X stage drive mechanism 52 and the white stage drive mechanism, and it is possible to move the camera section 10 that includes the image sensor 12a in the X axis direction and the Y axis direction using these drive mechanisms.

The cell observation device 1 of this embodiment also has the image sensor 12a, and it is possible to acquire images of cells (specimen 81) at a desired position. The cell observation device 1 camera can also perform live view display using images that have been acquired, and can store acquired images as still images or movies, and play back stored images later. The cell observation device 1 can also analyze images that have been acquired, and can perform various analysis such as counting a number of cells.

Also, with this embodiment, heat is generated if various members and circuits, such as the image sensor 12a, LD motor 51, X stage motor 53, Y stage motor 58, LED 13, LED drive circuit 59a etc. are operated. As a result, temperature of the cell observation device 1 and around the cell observation device inside the incubator 100 rises. Therefore, with this embodiment, operation time of these electronic components and circuits etc. is controlled based on the detection output of the device interior temperature sensor 43a. In addition to the device interior temperature sensor 43a, a device exterior temperature sensor may be provided, and measured temperature results from this temperature sensor also reflected.

Next, examples of the cell culture vessel 80 will be described using FIG. 4A to FIG. 4C. FIG. 4A shows an example of a flask-shaped vessel 82, and this vessel is disclosed in Japanese patent laid-open No. 2013-116073. With this example, the inside of the vessel main body section 82a is a cavity, and a locking section 82d that locks a lid 82c is formed on a neck section 82b that continues to this cavity. With this example a culture medium is held inside the vessel main body section 82a, and cells can be cultivated.

FIG. 4B shows an example of a dish-shaped vessel 83. This vessel 83 has a wall section formed around a bottom section, and the upper part is formed open. A standard size for the diameter of the dish-shaped vessel is 100 mm, but various sizes are available.

FIG. 4C shows an example of a well (micro) plate shaped vessel 84, and this vessel is disclosed in Japanese patent laid-open No. 2014-506799. With this example, 4×6=24 small cavities 84a are provided, and cells are cultivated by placing respective culture mediums in each cavity 84a. The example shown in FIG. 4C is a 24 well type vessel, but as well as this type there are vessels of varying well numbers, such as 6 wells and 12 wells.

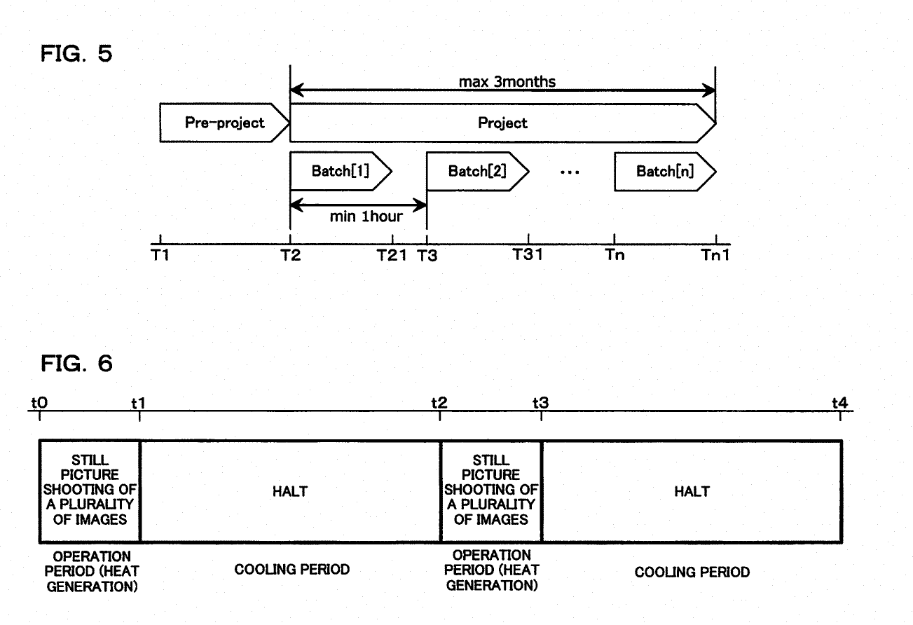

Next, cell cultivation and an overview of an operation to observe these cells will be described using FIG. 5. In FIG. 5, the horizontal axis represents the flow of time. From time T1 to time T2 a pre-project operation is performed, and from time T2 to time T3 a project operation is performed. The pre-project operation is an operation for performing preprocessing for a project operation. The pre-project operation performs setting for type of the cell culture vessel 80 and operations that will be performed in a project operation. The project operation will be described later.

In FIG. 5, with the project operation batch 1 (batch processing will be described later) is executed from time T2 to time T21, batch 2 is executed from time T3 to time T31, and after that up to batch n is executed at given time intervals. A time from commencement of a batch until commencement of the next batch is made constant (for example a minimum of about one hour), but may be appropriately changed to conform to cell cultivation observation interval. Also, a project operation period is, for example, a maximum of about three months, but this may also be changed to conform to the cell cultivation period.

Detailed operation of the batch processing shown in FIG. 5 will now be described using FIG. 6. Batch processing is processing for the camera section 10 to perform imaging of cells (specimen 81) in an order that has been set in advance and at positions that have been set in advance. As was described previously, the image sensor 12a, LED 13, and each of the motors and drive circuits, generate heat at the time of imaging, as a result of which temperature inside the cell observation device 1 rises. If the temperature within the cell observation device rises, temperature (air temperature) of a limited area surrounding the cell observation device 1, which is for maintaining the inside of the incubator 100 at a constant temperature, rises. Since the specimen 81 (cell) inside the cell culture vessel 80 is arranged on the top plate 40 of the cell observation device 1, heat is also transferred to the specimen 81. As a result temperature of the specimen 81 also rises, which has an adverse effect on the cells. With this embodiment therefore, once a given operating period has elapsed operation of the image sensor 12a etc. is stopped, and after a cooling period has elapsed the image sensor 12a etc. is operated again.

FIG. 6 shows one example of operation for batch processing, with the horizontal axis showing the flow of time. From time t0 until time t4 is one batch processing period. During the period from t0 to t1, a plurality of still images are taken. During this operation period (t0 to t1) the image sensor 12a etc. is in an operating state and so heat is generated. Once the operation period has elapsed and time t1 is reached sensors motors and circuits etc. of the image sensor 12a are stopped, and up until time t2 constitutes a cooling period. After that, the period from time t2 to time t3 constitutes an operation period, and from time t3 to time t4 constitutes a cooling period.

In FIG. 6, two operation periods and cooling periods are respectively provided. However, the number of operation periods and cooling periods may be appropriately selected depending on various conditions such as range of cell observation, heat generation amount of the image sensor 12a, etc. For example, at the time of taking pictures of the cell culture vessel 80, 50×20, or a total of 1000 still images, is set, with 200 pictures being taken in 10 minutes, and a cooling period of 30 minutes is set. In this case, still picture shooting of 1000 images is performed by repeating this cycle of shooting and cooling five times, and this is made a single batch operation.

Next, an example of this embodiment where there is a rise in internal temperature in the operation period and then a fall in internal temperature in a cooling period, will be described using FIG. 7. The horizontal axis shows time, and time t12 shows a time at which a standard time (for example 10 minutes) has elapsed. The vertical axis shows internal temperature (temperature equivalent to an extent of change from an initial temperature) and temperatures of threshold value H, threshold value 1 and threshold value L are shown by dashed lines. The threshold values H, 1 and L are appropriately and optimally set taking into consideration heat generation amount of the cell culture vessel 80. A plurality of threshold values H, 1 and L are stored in advance in the storage section 71 in accordance with type of cell culture vessel. The CPU 60 performs determination of temperature by reading out threshold values H, 1 and L corresponding the cell culture vessel, which is set (input) by the user, from the storage section 71.

In the example shown in FIG. 7, the temperature change curve L1 shows an example where threshold value H is reached at time t12 before time ts, corresponding to the standard time (for example, 10 minutes), is reached. In this case, standard time as a cooling time is made 30 minutes, for example. The temperature change curve L2 exceeds threshold value 1 at time t11 which is before time ts, and operation time extends until time t14. It should be noted that in this case also, cooling time is made the standard time (for example, 30 minutes). Temperature change curve L3 shows an example where threshold value 1 is reached at precisely time ts, and there is a transition to the cooling period at this time. It should be noted that in this case also, cooling time is made the standard time (for example, 30 minutes).

In the example shown in FIG. 7, temperature change curve L4 does not reach threshold value 1 at time ts, and there is a transition to the cooling period at time ts. In this case, since internal temperature has not become high the cooling period is made shorter than the standard time. Temperature change curve L5 does not exceed threshold value L even at time ts. In this case since internal temperature is low a cooling period is not provided.

It should be noted that as was described using FIG. 4, with this embodiment it is possible to use various cell culture vessels 80. Since amount of heat generated differs depending on the type of cell culture vessel 80, the temperature change curves shown in FIG. 7 will also differ. With this embodiment, therefore, threshold values are changed depending on the type of vessel.

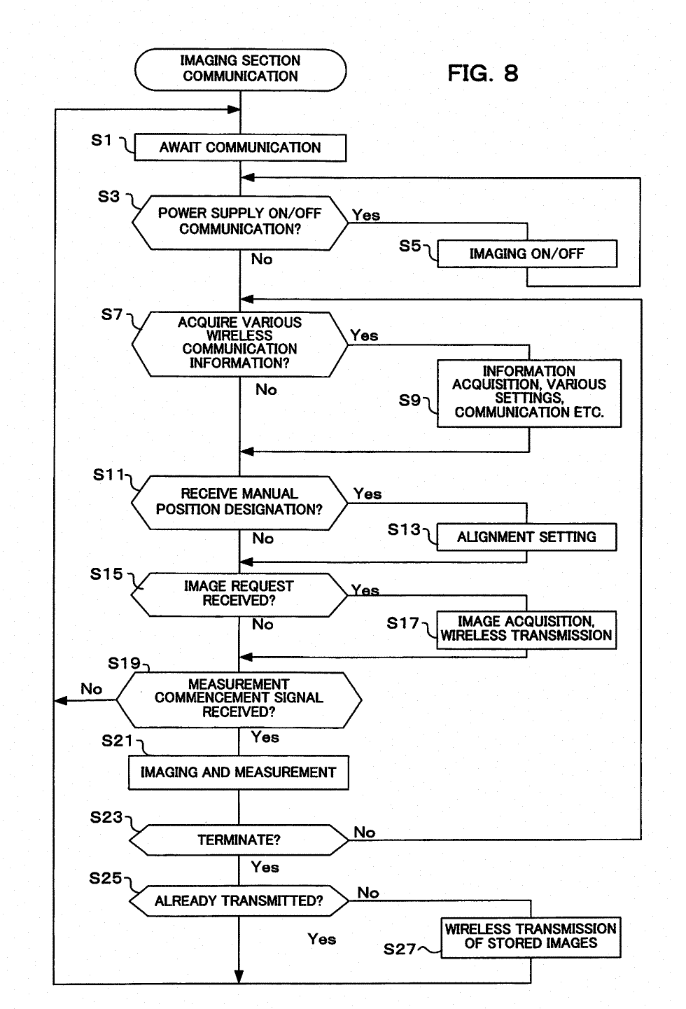

Next, operation of the cell observation device 1 will be described using the flowchart shown in FIG. 8. This flowchart is executed by the CPU 60 controlling each of the sections within the cell observation device 1 in accordance with program code that has been stored within the storage section 71. In this flowchart, description will be given for when the user controls the cell observation device 1 using the cell observation auxiliary control unit 220. However, the cell observation device 1 may also be controlled using the cell observation main control unit 210.

If the flowchart for imaging section communication shown in FIG. 8 is commenced by a power supply being switched on or the like, first a communication stand by state is entered (S1). Here, commencement of communication from the cell observation auxiliary control section 220 is awaited. Specifically, the cell observation device 1 is arranged within the incubator 100, separated from external sections. When the user issues a command to this cell observation device 1 the cell observation auxiliary control section 220 may be operated. This step is a state of awaiting receipt of a control signal based on this operation, using wireless communication.

Next, it is determined whether or not power supply on/off communication has been performed (S3). As was described previously, with this embodiment power supply for the cell observation device 1 is supplied using a battery, and so in order to prevent consumption of the power supply batteries it is possible for the user to perform a power supply on or power supply off instruction from the cell observation auxiliary control section 220.

If the result of determination in step S3 is that there has been power supply on/off communication, imaging on/off processing is performed (S5). Here, when the power supply has been turned on, power for each section within the CPU 60, the camera section 10, and the X/Y stage section 50 is turned on. On the other hand, when the power supply has been turned off, power for each section within the CPU 60, the camera section 10 and the X/Y stage section 50 is turned off. Power supply on/off for each of the other sections may also be carried out in tandem with power supply on/off of the camera section 10. However, even if the power supply is off, a minimal power supply is provided in order to execute functions for determining instructions from the cell observation auxiliary control section 220. For example, power is supplied for the basic sections of the CPU 60 and for the wireless communication device 18. As a result of this power supply control it becomes possible to reduce wasteful energy consumption.

If the result of determination in step S3 is not power supply on/off communication, it is determined whether or not various wireless communication information has been acquired (S7). If the user performs various settings by operating the cell observation auxiliary control section 220, this setting information is transmitted from the communication section 228 of the cell observation auxiliary control section 220 by means of wireless communication. Information that is necessary to imaging is also transmitted by wireless communication from the communication section 228. For example, as information that is transmitted here there is information relating to transmission destination of image data, conditions for at the time of imaging, various parameters, and measurement conditions for when measuring the specimen 81 etc. In this step, determination is based on whether or not these items of information and settings have been received by the wireless communication device 18 within the cell observation device 1.

If the result of determination on step S7 is that various wireless communication information has been acquired, information acquisition, various setting and communication etc. are performed (S9). In this step various settings within the cell observation device 1 are performed based on the various information and settings that have been acquired by the wireless communication device 18. In these steps S5, S9 and S13 the pre-project operation that was shown in FIG. 5 is executed. Details of the pre-project operation will be described later using S61 to S79 in FIG. 10.

Once the information acquisition, various settings and communication etc., have been performed in step S9, or if the result of determination in step S7 was that various information has not been acquired, it is next determined whether or not a manual position designation has been received (S11). There may be cases where the user wishes to observe images at a position that has been designated during preparations for measurement of the specimen 81 within the cell culture vessel 80, or during measurement itself. In such a case the user can designate imaging position by operating the cell observation auxiliary control section 220. In this step, it is determined whether or not wireless communication for performing this manual position designation has been received.

If the result of determination in step S11 is that manual position designation has been received, alignment is performed (S13). Here, a description is given using the example shown in FIG. 2, where the movement control section 33 moves the camera section 10 to a manually designated position, by controlling drive of the Y actuator 31a and the X actuator 31b to the designated manual positions that has been received by means of wireless communication. Also, in the description given using the example shown in FIG. 3, the CPU 60 issues commands to the X/Y stage section 50 so as to move the camera section 10.

If alignment setting has been performed in step S13, or if it has been determined that manual position designation has not been received, it is next determined whether or not an image request has been received (S15). There may be cases where the user, while preparing for measurement or during measurement, wishes to observe images at the manual position that has been designated. In such cases, an image request is transmitted by operating the cell observation auxiliary control section 220. There may also be cases where during measurement the user wishes to confirm images that have been captured so far, and in this type of situation also an image request is transmitted by operating the cell observation auxiliary control section 220. In this step, therefore, it is determined whether or not an image request signal has been received from the cell observation auxiliary control section 220.

If the result of determination in step S15 is that there is an image request signal, image data is acquired and wireless communication is performed (S17). In this case, imaging is performed at the point where alignment was carried out in step S13, and that image data is transmitted to the cell observation auxiliary control section 220. In the event that, during measurement, there has been a request to transmit image data that has been acquired so far, image data that has been stored in the storage section 71 is read out and that image data is transmitted to the cell observation auxiliary control section 220. It should be noted that in step S9, in the event that a section other than the cell observation auxiliary control section 220 is designated as the transmission destination for the image data, the image data is transmitted to that designated transmission destination. Also, in a case where image data has been transmitted, a transmitted flag is set for the transmitted image data.

If image data has been acquired and wireless communication performed in step S17, or if the result of determination in step S15 is that an image request has not been received, it is next determined whether or not a measurement commencement signal has been received (S19). When commencing measurement, such as counting a number of cells of the specimen 81 within the cell culture vessel 80, the user instructs that fact to the cell observation device 1 by operation of the cell observation auxiliary control section 220. Here it is determined whether or not a measurement commencement signal to instruct commencement of this measurement has been received. If the result of this determination is that a measurement commencement signal has not been received, processing returns to step S1 and the previous operations are executed.

On the other hand, if the result of determination in step S19 is that a measurement commencement signal has been received, imaging and measurement are commenced (S21). Here, measurement is performed under shooting conditions that have been set, and then results stored, in accordance with a positioning program that has been set and stored. Also, if measurement is interrupted and then restarted, imaging and measurement are restarted from the interrupted position. It should be noted that from a yes determination in step S19 to step S27 is equivalent to the duration of each project operation of FIG. 5.

Specifically, the camera section 10 sequentially performs imaging (still picture shooting) in accordance with positions and shooting conditions that have been designated by the movement patterns that have been stored in the storage section 71, and the image data that has been acquired is stored in the storage section 71. At the time of storage, various data such as position of the camera section 10, time, shooting conditions etc. is appended as tags. This imaging involves a read out step of reading out position of the camera section 10 and control data (for example, movement patterns) for controlling imaging conditions at the time of imaging using the camera section 10, an imaging step of acquiring image data of a physical object including the specimen 81 using the camera section 10, and a position changing step of changing imaging position of the camera section 10 based on control data.

In this way, with this embodiment, since shooting position and shooting conditions are set in accordance with various control data that has been stored in storage section 71, during measurement it is not necessary for the cell observation auxiliary control section 220 to carry out frequent communication with the camera section 10, and wasteful energy consumption for communication is suppressed. Also, as shooting conditions, it may be possible to set lighting conditions with the LED 13 etc.

Also, in step S21, batch processing of the project operation that was shown in FIG. 5 and FIG. 6 is repeatedly performed a given number of times. Specifically, performing of still image shooting for a plurality of images (operation period) and stopping the still picture shooting (cooling period) is repeated, to execute a single batch processing. Detailed operation of this project operation will be described later using S83 to S91 in FIG. 10.

Also, in step S21 if image data is acquired by performing still picture shooting, a number of cells of the specimen 81 is counted by analyzing this image data, and stored. Counting of the cells of the specimen 81 is performed by detecting edges and contours within the image data, and using various known procedures such as extracting individual specimens 81 (cells). The number of cells of the specimen 81 is appended as a tag to the image data and stored in the storage section 71.

There may also be cases where measurement is interrupted, such as when a manual position designation is received or an image request signal is received during measurement. In this type of situation, processing that has been requested is executed, and after that, when restarting the measurement, measurement commences from the interrupted position. Therefore, position when the interrupt occurred, and sequence number for the movement pattern etc. are stored in the storage section 71.

If imaging and measurement have been carried out, it is next determined whether or not the imaging and measurement have been completed (S23). Here, it is determined whether or not imaging and measurement have been completed in accordance with all movement patterns that are stored in the storage section 71. If the result of this determination is that imaging and measurement have been completed, processing returns to step S7 and the previous operations are executed. In the event that the user operates the cell observation auxiliary control section 220 during measurement and various settings, designation of manual position or an image request are performed, processing is executed in accordance with these instructions.

If the result of determination in step S23 is that imaging and measurement are complete, it is determined whether or not there has been transmission (S25). Here it is determined whether or not image data with time and data and coordinates attached that has been stored in the storage section 71 has been transmitted to the cell observation auxiliary control section 220. At this time, image data that was already transmitted in step S17 will overlap. Therefore, in order to transmit image data that has not yet been transmitted determination as to whether or not data has been transmitted is performed for each image data. Accordingly, with this embodiment, depending on imaging position and imaging conditions that have been stored in the storage section 71, the wireless communication device 18 will have a time for carrying out communication for positioning and shooting control of the camera section 10, and a time for communication of information that has been acquired by imaging.

If the result of determination in step S25 is that there are images that have not already been transmitted, stored images are subjected to wireless transmission (S27). Here, among the images that were captured in step S21, images that were not transmitted in step S17 are subjected to wireless transmission.

If stored images have been transmitted in step S27, or if the result of determination in step S25 was that there were already transmitted images, processing returns to step S1 and the previously described operations are executed.

Also, with the imaging section communication flow of this embodiment, signals corresponding to image data (refer, for example, to S17 and S27), and signals for changing position of the camera section 10 (refer, for example, to S13, S15, S19 and S21) are interchanged between the wireless communication device 18 within the cell observation device 1 and the communication section 228 within the cell observation auxiliary control section 220. In this way, movement of the camera section 10 is controlled together with the interchange of image data using a single communication line. This means that this is possible to simply perform imaging and measurement of a measured physical object even if the cell observation device 1 is isolated within a chamber such as an incubator 100.

Also, if a measurement commencement signal is received (refer to S19), imaging is performed at sequential measurement positions in accordance with given sequence numbers, in accordance with a movement pattern that has been stored in the storage section 71. As a result, if a movement pattern is decided upon in advance, it is possible to carry out imaging and measurement automatically. It is also possible to observe a specimen by interrupting imaging during measurement (refer, for example, to S11-S15). Also, in the case of being interrupted during measurement, measurement is restarted from the interrupted position (refer, for example, to S21).

It should be noted that shooting conditions for visual observation of the specimen 81 within the cell culture vessel 80 by the user (such as aperture, shutter speed, ISO sensitivity), are not necessarily the same as the shooting conditions for measurement (counting) of the specimen 81. There are also situations where shooting conditions that are suitable for visual observation (live view display) and shooting conditions that are suitable for image storage are different. Shooting may therefore be respectively performed with a plurality of shooting conditions at a single location, to acquire image data. Also, only in a situation where the user has requested images for observation (refer, for example, to S15), shooting may be performed using shooting conditions for visual observation, and when performing shooting for measurement in accordance with a movement pattern shooting may be performed using shooting conditions for measurement. Also, as shooting conditions, there are shooting conditions suitable for change in illuminated state using the LED 13, shooting conditions suitable for collection conditions and growth conditions of cells, focus conditions that are suitable for positions of cells etc., and shooting may be performed under numerous conditions.

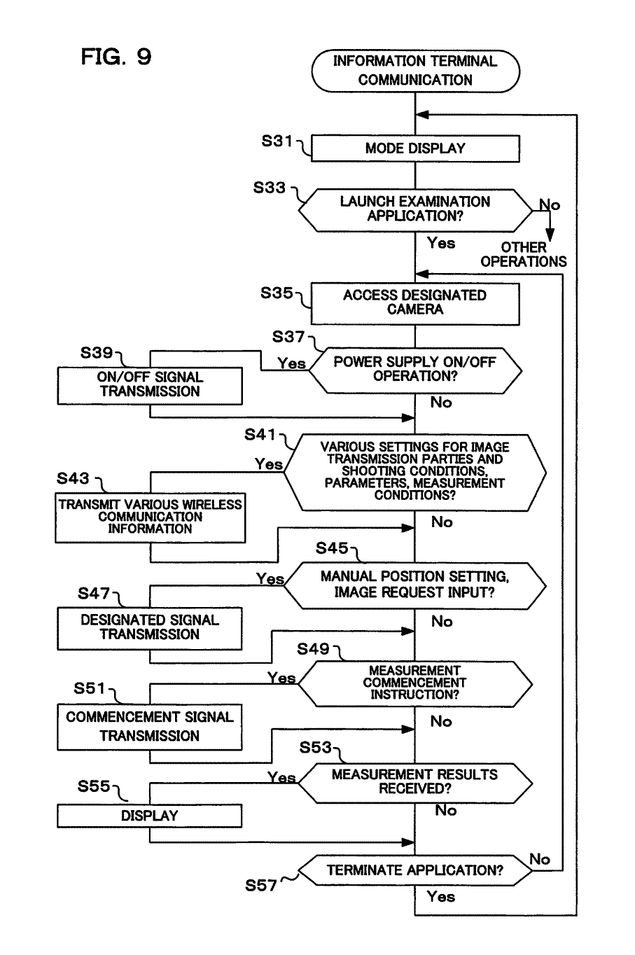

Next, operation of the cell observation auxiliary control unit 220 will be described using the flowchart for information terminal communication shown in FIG. 9. This flowchart is implemented by the control section (CPU etc.) within the cell observation auxiliary control unit 220 controlling each of the sections within the cell observation auxiliary control unit 220 in accordance with program code that has been stored within the storage section. In this flowchart, description will be given for when the user controls the cell observation device 1 using the cell observation auxiliary control unit 220, but control may also be performed using the cell observation main control unit 210.

If the flow for information terminal communication is entered, first mode display is performed (S31). Here, a mode of the cell observation auxiliary control unit 220 is displayed on the display section 229. For example, if the cell observation auxiliary control unit 220 doubles as a smartphone, there are mobile phone mode, mail mode etc.

Once mode display has been carried out, it is next determined whether or not to launch an examination application (S33). Here it is determined whether or not an application for examining (measuring) to count a number of cells of the specimen 81 (hereafter referred to as “examination application”) will be launched. For example, an examination application icon is displayed in step S31, and if a touch operation is performed on this icon it is determined that the examination application will be launched. As well as the examination application there may also be application to analyze images of cells. Also, as an application selection method, besides a touch operation, it may be determined to launch the application if a cursor is moved to select an icon, and it may be determined to launch the application if a dedicated button is operated. If the result of this determination is not to launch the examination application, then other operations, for example, in the case of a smartphone, mobile phone operations and mail operations, are performed.

If the result of determination in step S33 is to launch the examination application, then a designated camera is accessed (S35). Here, a camera that was designated by the cell observation auxiliary control unit 220 (the cell observation device 1 with the example of FIG. 1 and FIG. 2) is accessed. Specifically, communication is performed from the communication section 228 of the cell observation auxiliary control unit 220 to the wireless communication device 18 of the cell observation device 1.

Next it is determined whether or not an imaging on/off operation has been performed (S37). The cell observation device 1 is arranged in a chamber such as an incubator, a specimen 81 inside the cell culture vessel 80 is examined, and supply of power is received from the power supply battery. In order to prevent power consumption, it is possible for the user to instruct power supply on/off for the cell observation device 1 from the cell observation auxiliary control unit 220. Here, it is determined whether or not a power supply on/off operation was performed using the cell observation auxiliary control unit 220.

If the result of determination in step S37 is that a power supply on/off operation has been performed, an on/off signal is transmitted (S39). Here, a power supply on/off signal is transmitted from the communication section 228 of the cell observation auxiliary control unit 220 to the wireless communication device 18 of the cell observation device 1. The cell observation device 1 executes a power supply on/off operation (refer to S5 in FIG. 8) if this signal is received (refer to S3 in FIG. 8).

If the on-off signal has been transmitted in step S39, or if the result of determination in step S37 is that a power supply on/off operation is performed, it is next determined whether or not to carry out various settings, such as for image transmission parties, shooting conditions, parameters and measurement conditions etc. (S41). It is possible to designate destinations for transmission of image data that has been captured by the cell observation device 1, and various information that is attached to the image data as tags (time and date information, position information, measurement (examination) result information). Transmission destinations are not limited to the cell observation main control unit 210 or the cell observation auxiliary control unit 220, and may be other information terminals etc.

Also, shooting conditions (focus position, aperture value, shutter speed value, ISO sensitivity value, switching of image processing including enhancement of edges, contrast and color etc., and brightness, pattern and wavelength of illumination) for when the cell observation device 1 is imaging, may also be set in step S39. Parameters and measurement conditions etc. may also be set in this step. It may also be possible to set patterns besides a default pattern for a movement pattern. With this embodiment default patterns are stored in the storage section 71 of the cell observation device 1 and in the storage section of the cell observation auxiliary control unit. In this step S41, it is determined whether or not an operation has been performed in order to perform these various settings.

If the result of determination in step S41 is that operations for various settings have been performed, various wireless communication information is transmitted (S43). Here, operated information is transmitted from the communication section 228 to the wireless communication device 18 of the cell observation device 1 based on the determination in step S41 (refer to S7 and S9 in FIG. 8).

If various wireless communication information has been transmitted in step S43, or if the result of determination in step S41 was that an operation for various settings was not performed, it is next determined whether or not manual position setting or an image request have been input (S45). As was described previously, if the user designates position of the camera section 10 when preparing for measurement or during measurement, and they wish to observe images that have been acquired with the camera section 10, it is possible to perform designation from the cell observation auxiliary control unit 220. In this step, it is determined whether or not these operations have been performed.

It should be noted, regarding position designation of the camera section 10, that designation may be by absolute position, such as (x, y) coordinates, and may be designation of movement of the camera section 10 by relative positional designation in a horizontal direction and vertical direction, while observing an image. Besides this it is also possible to control movement in accordance with operation amount of a touch panel, switch or dial of the operation section, and to determine a typical observation point and designate movement to that location.

If the result of determination in step S45 is that manual position setting or an image request have been input, designation signals are transmitted (S47). Here signals corresponding to operations in step S45 are transmitted from the communication section 228 to the wireless communication device 18 of the cell observation device 1 (refer to S11-S17 in FIG. 8).

If designation signals have been transmitted in step S47, or if the result of determination in step S45 was that manual position setting or an image request were not input, it is next determined whether or not to perform a measurement commencement instruction (S49). In this step it is determined whether or not the user has instructed measurement commencement. As described previously, imaging is performed while sequentially moving the camera section 10 in accordance with a movement pattern, and measurement involves counting the specimen 81 etc. based on image data that has been formed. Instruction of measurement commencement may be performed by a touch operation of a measurement commencement icon that has been displayed on the display section 229 of the cell observation auxiliary control unit 220.

If the result of determination in step S49 is that there has been a measurement commencement instruction, a commencement signal is transmitted (S51). Here, a measurement commencement signal is transmitted from the communication section 228 to the wireless communication device 18 of the cell observation device 1 (refer to S19 and S21 in FIG. 8).

If a commencement signal has been transmitted in step S51, or if the result of determination in step S49 is that there was not a measurement commencement instruction, it is next determined whether or not measurement results have been received (S53). Here, images acquired using the camera section 10 that have been transmitted are displayed on the display section 229. Display of measurement results of the specimen 81 etc. is also performed.

If display has been carried out in step S55, or if the result of determination in step S53 was that measurement results were not received, it is determined whether or not to terminate the application (S57). Here it is determined whether or not an instruction to terminate operation of the examination application, that was launched in step S33, has been issued. If the result of this determination is not to terminate the examination application, processing returns to step S35, while if the result of determination is to terminate the examination application processing returns to step S31.

In this manner, in the flow for information terminal communication, if various setting operations are performed in the cell observation auxiliary control unit 220 to move the camera section 10, signals are transmitted by means of the communication section 228 to the wireless communication device 18 of the cell observation device 1 based on settings (for example, S39, S43, S47, S51). Also, images that have been acquired by the camera section 10 are transmitted from the wireless communication device 18 of the cell observation device 1 to the communication section 228 (S55). In this way, even if the cell observation device 1 is isolated inside a sealed chamber such as an incubator, it is possible to transmit instructions from the cell observation auxiliary control unit 220, and it is possible to receive image data from the cell observation device 1. This means that it is possible to perform imaging and measurement of a measured physical object simply.

Next, processing for the pre-project operation and batch operation shown in FIG. 5 will be described using the flowchart shown in FIG. 10. This flowchart (also including FIG. 11 which will be described later) is implemented by the CPU 60 controlling each of the sections within the cell observation device 1 in accordance with program code that has been stored within the storage section 71. It should be noted that the previously described pre-project operation (S61 to S79) is performed in steps S5, S9 and S13 (refer to FIG. 8), and the project operation (S81 Yes to S91) is performed in step S21 (refer to FIG. 8).

If the flow for the pre-project operation of FIG. 10 is entered, first initialization of the cell observation device 1 is performed (S61). Here, electrical initialization is performed, such as performing initialization of various data that has been stored in the storage section 71 within the cell observation device 1. Operation for initialization of the cell observation device is processing corresponding to the imaging on operation of S5 in FIG. 8.

Next, the setting of cell type is performed (S63). Here, setting of the type of cells to be filled into the cell culture vessel 80 is set by the user operating a user interface (for example, character input etc. using touch operation) of the cell observation auxiliary control unit 220.

Next, setting for the culture vessel is performed (S65). As was described previously using FIG. 4, there are various types of cell culture vessel 80. Here, what type of cell culture vessel 80 will be used is set by the user operating a user interface (for example, character input etc. using touch operation) of the cell observation auxiliary control unit 220 (or the cell observation main control unit 210).

Once setting of culture vessel has been performed, next observation range (region) setting is performed (S67). Here, observation range of the specimen 81 is set based on a movement pattern etc. that was acquired in step S9.

Specifically, movement range of the camera section 10 by the X stage drive mechanism 52 and the Y stage drive mechanism 59 is set. As was described previously, since the size of the cell culture vessel differs depending on its type, culture medium surface area also differs, and observation range also differs. For this reason, observation range (region) that was stored in advance in the storage section or storage section 71 may be set in accordance with the type of cell culture vessel that has been designated. Movement patterns (imaging position and shooting order) for the camera section 10 may also be stored in advance in the storage section or storage section 71 in accordance with the type of cell culture vessel, and movement pattern may be set in accordance with the cell culture vessel setting.

If setting of observation range has been performed, next calculation of a number [m] of still images to be taken is performed (S69). As well as observation range, a movement pattern may also include information on still image shooting position. In this step, a number [m] of images for still picture shooting is calculated based on the movement pattern.

Once calculation of a number of still images to be taken has being performed, next setting for batch operation is performed (S71). As was described previously using FIG. 6, a batch operation is performed to take into consideration a cooling period. Specifically, still image shooting is not performed at once for all shooting positions within an observation range, and instead still image shooting is performed for shooting positions with the cooling period interposed so that temperatures of the cell observation device 1 and temperature around the cell observation device 1 within the incubator 100 are kept in a given range. If a single batch operation is completed, still picture shooting for all shooting positions is completed. In this step, setting of a batch operation that includes shooting positions corresponding to an operation period, and a cooling period, is performed.

If a batch operation has been performed, next project period setting is performed (S73). The project period is a period for cultivating cells. The user sets a period for observing the specimen 81 (cells) by operating a user interface of the cell observation auxiliary control unit 220.

If project period setting has been performed, next batch interval setting is performed (S75). Still picture shooting for all shooting positions is completed with the above described single batch operation. The batch interval is a time from commencement of a batch operation until commencement of the next batch operation. Specifically, batch interval means what time interval shooting of the specimen 81 is performed at. The user sets the batch interval by operating a user interface of the cell observation auxiliary control unit 220.

If batch interval setting has been performed, calculation of a number of batches [n] is performed (S77). The number of batches [n] is calculated based on the project period that was set in step S73, and on the batch interval that was set in step S75. Once the number of batches has been calculated, a variable [i] for counting batch processing is set to 1 (i=1) (S79).

If the variable i has been set to 1, it is next determined whether or not there is project commencement (S81). As was described previously, if the cell observation device 1 receives a measurement commencement signal in step S19 imaging is commenced, and the project operation show in FIG. 5 is commenced. In this step determination is based on whether or not this measurement commencement signal has been received.

If the result of determination in step S81 is project commencement, a project operation is executed. First, operation initialization of the cell observation device 1 is performed (S85). Here, mechanical initialization is performed, such as moving the camera section 10 within the cell observation device 1 to an initial position. Electrical initialization is also performed, such as performing initialization of various data that have been stored in the storage section 71.

If operation initialization of the cell observation device has been performed, next batch [i] is executed (S87). Here, still picture shooting for a plurality of images (operation period) and operation halt (cooling period), that were shown in FIG. 6, are repeated, in accordance with the settings of steps S71, S75 and S77, to perform still picture shooting at each shooting position. Detailed operation of this batch [i] will be described later using FIG. 11.

Once batch [i] has been performed, variable i is incremented by 1 (i=i+1) (S89). Next, it is determined whether or not the variable i has become n+1 (S91). Here it is determined whether or not the variable i that was incremented by 1 in step S89 has reached a value resulting from adding 1 to the number of batches n that was calculated in step S77. If the result of this determination is that the variable i has not reached the number of batches n, the batch operation is repeated until the number of batch operations that have been processed in step S87 reaches n. If the result of determination in step S91 is that the variable i has reached the number of batches n, the originating flow is returned to.

Next, detailed operation of the batch [i] of step S87 will be described using the flowchart shown in FIG. 11.

If the flow for batch [i] is entered, first temperature is measured (S101). Here, temperature inside the cell observation device 1 is measured using the device interior temperature sensor 43a. Once temperature is measured, initial temperature temp0 is set to temp (S103). Specifically, the temperature temp was measured in step S101 is set as the initial temperature temp0.

Next, a timer 1 is initialized and a clocking operation is commenced (S105). In this embodiment, for each batch operation a standard time for still picture shooting is made 10 minutes, and a cooling time is made 30 minutes. The timer 1 performs clocking operations for these times.

If the timer 1 has been initialized and a clocking operation has been commenced, next an X-Y stage drive operation is performed (S107). Here, the camera section 10 is moved to a shooting position that has been designated by the movement pattern etc., using the X stage drive mechanism 52 and the Y stage drive mechanism 59.

If the X-Y stage drive operation has been carried out, next AF (Auto Focus) is performed (S109). Here, the lens 11a is moved to an in focus position using a contrast method, for example, based on image data that has been acquired by the image sensor 12a. Specifically, the image processing section 63 calculates an AF evaluation value (contrast value) based on image data, and the LD motor control section 61 performs focus adjustment by controlling the LD motor 12b so that the AF evaluation value becomes a peak. It should be noted that the AF is not limited to a contrast method and may be performed using another method, such as a phase difference method, etc.

Once AF has been performed, next still picture shooting is performed (S111). In step S107 the camera section 10 is moved to a shooting position that has been designated, and after focusing of the lens 10a in step S109 the still picture shooting is carried out in this step. In this still picture shooting, image data is acquired by the image sensor 12a, image processing is performed by the image processing section 63, and image data is stored in the storage section 71.

If still picture shooting has been performed, 1 is subtracted from the variable m for number of images to be taken (S113). It is then determined whether or not the variable m that has been subjected to subtraction is 0 (S115). Here it is determined if the variable m for number of images to be taken that was set in step S69 (FIG. 10) is 0, in other words, whether or not shooting has been completed for the number of images for still picture shooting that was set in advance. If the result of this determination is that the variable m is 0, then the intended still picture shooting has been completed, and so the original flow is returned to (S89 in FIG. 10).

On the other hand, if the result of determination in step S115 is not that m=0, measurement is performed (S117). In step S101, initial temperature inside the cell observation device 1 is measured. Due to heat generation with operation of various electronic devices such as the image sensor 12a, motors 53 and 58 etc., temperature inside the cell observation device 1 and temperature around the cell observation device 1 inside the incubator 100 rise. Temperature is therefore measured similarly to in step S101. At this time, the temperature that has been measured is made temp.

It should be noted that instead of this temperature inside the cell observation device 1 that has been measured, temperature of the specimen 81 (cells) within the cell culture vessel 80 may be estimated, and this estimated temperature made temp. Temperature of the specimen 81 (cells) within the cell culture vessel 80 is estimated to be a temperature between the temperature inside the cell observation device 1 and a constant temperature within the incubator 100 that has been set. Taking this type of situation into consideration, it is preferable to estimate temperature of the specimen 81 (cells) using a formula for estimation that has been stored in advance in the storage section 71.

Once temperature measurement has been performed, it is next determined whether or not temp-temp0≥threshold value H (S119). The temperature temp is the most recent temperature that was measured in step S117, while temperature temp0 is the initial temperature that was measured in step S101. It is determined whether a difference between the two is larger than the threshold value H shown in FIG. 7.

If the result of determination in step S119 is that temp-temp0 is greater than or equal to threshold value H, cooling is performed for 30 minutes (S127). This case corresponds to temperature change curve L1 in FIG. 7, and since the most recent temperature is a considerably high temperature the still picture shooting operation is immediately halted and each of the sections within the cell observation device 1 is cooled. By stopping operation of each of the sections within the cell observation device 1, temperature within the cell observation device 1 and temperature around the cell observation device 1 within the incubator 100 gradually falls. That is, the temperature of the specimen 81 within the cell culture vessel 80 becomes close to an intended set temperature within the incubator 100. Then, once the cooling period of 30 minutes has elapsed, step S105 is returned to.

On the other hand, if the result of determination in step S119 is that temp-temp0 is less than threshold value H, it is next determined whether or not the timer 1 has reached 10 minutes (S121). Here it is determined whether or not the timer 1 that started clocking operation in step S105 has reached 10 minutes. If the result of this determination is that 10 minutes has not elapsed, processing returns to step S107.

On the other hand if the result of determination in step S121 is that 10 minutes have elapsed, it is determined whether or not threshold value 1>temp-temp0≥threshold value L (S123). The temperature temp is the most recent temperature that was measured in step S117, while temperature temp0 is the initial temperature that was measured in step S101. It is determined whether or not a difference between the two temperatures is between threshold value L and threshold value 1 shown in FIG. 7.

If the result of determination in step S123 is not that threshold value 1>temp-temp0≥threshold value L, processing returns to S107. This case corresponds to temperature change curve L2 or L5 in FIG. 7. In the case of corresponding to temperature change curve L2, a cooling operation of 30 minutes is commenced from the point in time where the difference exceeded threshold value H in step S119. Also, in the case of corresponding to temperature change curve L5, then since the temperature rise within the cell observation device 1 and the temperature rise around the cell observation device 1 within the incubator 100 are quite low, it can be considered that temperature of the specimen 81 (cells) within the cell culture vessel 80 is being kept at about the intended set temperature for the incubator 100. Accordingly, since cooling is not particularly required still picture shooting continues without any further action.

On the other hand, if the result of determination in step S123 is that threshold value 1>temp-temp0≥threshold value L, shortened cooling is performed (S125). This case corresponds to temperature change curve L4 in FIG. 7. Since the temperature change is between threshold value 1 and threshold value L, the still picture shooting operation is halted and cooling is performed for only a short time. The cooling period may be a time that is shorter than 30 minutes, taking into consideration threshold value 1 and threshold value L. Once the cooling period has elapsed, step S105 is returned to.

In this way, in the flow for batch [i], the time for cooling commencement (S119→S127, S123→S125) is changed in accordance with temperature within the cell observation device 1 and temperature around the cell observation device 1 within the incubator 100, and the cooling period is changed (S125, S127). This means that even if there is change in temperature of the specimen 81 (cells) due to operation of the cell observation device 1 within the incubator 100, it is possible to keep this temperature change within a fixed range, and it is possible to provide an optimal environment for cell cultivation.

As has been described above, with one embodiment of the present invention, shooting position of an imaging section is changed (refer, for example, to S107 in FIG. 11), temperature inside the observation device is measured (refer, for example, to S101 and S117 in FIG. 11), and information about a vessel in which a sample is arranged is input (refer, for example, to S65 in FIG. 10). Also, when controlling shooting position of the imaging section and operation of the imaging section, an operation sequence, in which change of imaging position of the imaging section and imaging operation of the imaging section are repeated, is executed (refer, for example, to S107 to S111 in FIG. 11). The operation sequence is then stopped if a measured temperature or an estimated temperature exceed a given threshold value, and there is a transition to a halt sequence where a state in which change of a shooting position of the imaging section and the imaging operation are not executed continues for a given time (refer, for example, to S119 Yes→S127, S123 Yes→S125, in FIG. 11). As a result it is possible to acquire images using the imaging section while maintaining temperature within a given range, taking into consideration temperature rise that affects the sample (cells). It is also possible to perform more highly accurate temperature measurement if threshold values or given times are changed based on information about a vessel.

It should be noted that with the one embodiment of the present invention, 10 minutes has been set as the standard time shown in FIG. 7 (refer to S121 in FIG. 7), and 30 minutes has been set as the cooling period of FIG. 7 (refer to S127 in FIG. 11). However, these times are merely examples, and may be appropriately changed in accordance with characteristics of the incubator, cells, cell culture vessel, cell observation device, etc.

Also, description has been given of an example where three threshold values, namely threshold value L, threshold value 1 and threshold value H, have been adopted in FIG. 7 and FIG. 11. However, it is possible to have only two threshold values, namely threshold value L and threshold value H, and to have control such that cooling is immediately performed if the threshold value H is reached, and cooling is performed if temperature becomes threshold value L or higher 10 minutes after that, etc. It is also possible to perform control by further increasing threshold values.

Also, each of the sections within the LD motor control section 61 within the CPU 60 have been implemented in a software manner using programs, but some or all of these sections may be constructed as hardware circuits separate to the CPU 60, and may also be realized as a separate CPU. It is possible for these sections to have a hardware structure such as gate circuits generated based on a programming language that is described using Verilog, and also to use a hardware structure that utilizes software such as a DSP (digital signal processor). Suitable combinations of these approaches may also be used.

Also, with the one embodiment of the present invention description has been given of an example where the target of measurement is a specimen 81 (cells) cultivated in a culture medium inside a vessel 80. However, this is not limiting and it is possible to adopt this embodiment as long as it is with an object section when performing measurement while moving an imaging section within a given range. The communication method also does not need to be limited to wireless communication, and wired communication may also be used. Besides this, this embodiment can be utilized in any remote shooting examination system, device or method that carries out combination of quality examination of products, quality examination of parts and packages, and movement control and shooting control.

Also, among the technology that has been described in this specification, with respect to control that has been described mainly using flowcharts, there are many instances where setting is possible using programs, and such programs may be held in a storage medium or storage section. The manner of storing the programs in the storage medium or storage section may be to store at the time of manufacture, or by using a distributed storage medium, or they be downloaded via the Internet.

Also, with the one embodiment of the present invention, operation of this embodiment was described using flowcharts, but procedures and order may be changed, some steps may be omitted, steps may be added, and further the specific processing content within each step may be altered. It is also possible to suitably combine structural elements from different embodiments.

Also, regarding the operation flow in the patent claims, the specification and the drawings, for the sake of convenience description has been given using words representing sequence, such as “first” and “next”, but at places where it is not particularly described, this does not mean that implementation must be in this order.

As understood by those having ordinary skill in the art, as used in this application, ‘section,’ ‘unit,’ ‘component,’ ‘element,’ ‘module,’ ‘device,’ ‘member,’ ‘mechanism,’ ‘apparatus,’ ‘machine,’ or ‘system’ may be implemented as circuitry, such as integrated circuits, application specific circuits (“ASICs”), field programmable logic arrays (“FPLAs”), etc., and/or software implemented on a processor, such as a microprocessor.

The present invention is not limited to these embodiments, and structural elements may be modified in actual implementation within the scope of the gist of the embodiments. It is also possible form various inventions by suitably combining the plurality structural elements disclosed in the above described embodiments. For example, it is possible to omit some of the structural elements shown in the embodiments. It is also possible to suitably combine structural elements from different embodiments.

What is claimed is:

1. An observation device that has an image sensor that forms images of a specimen, comprising:
an imaging position change circuit that changes imaging position of image sensor,
a temperature sensor that measure temperature of the inside of the observation device and outputs results of this measurement as a measured temperature, and
a controller that controls operation of the imaging position change circuit and the image sensor, wherein
the controller
causes execution of an operating sequence that repeatedly executes change of imaging position of the image sensor by the imaging position change circuit and an imaging operation of the image sensor,
causes transfer to a halt sequence where the operating sequence is stopped if the measured temperature exceeds a given threshold value, and where a state in which change of imaging position of the image sensor and an imaging operation are prohibited continues for a given time, and
repeatedly executes the operating sequence and the halt sequence.

2. The observation device of claim 1, wherein:
as the threshold value there is a first threshold value, and as the given time there are a first given time and a second given time, and
the controller
in the event that the measured temperature does not exceed the first threshold value in a period from commencement of the operation sequence until the second given time has elapsed, continues the operation sequence even after the second given time has elapsed, and
in the event that the measured temperature exceeds the first threshold value in a period from commencement of the operation sequence until the second given time has elapsed, makes a transition to the halt sequence where the operation sequence is stopped, and continues the halt sequence for the first given time.

3. The observation device of claim 1, wherein:
as the threshold value there is a first threshold value, a second threshold value and a third threshold value, and as the given time there are a first given time and a second given time, and
the controller
in the event that the measured temperature has exceeded the second threshold value in a period from commencement of the operation sequence until the second given time has elapsed, and the measured temperature has not exceeded the third threshold value that is larger than the second threshold value and smaller than the first threshold value, stops the operation sequence, makes a transition to the halt sequence, and continues the halt sequence for a third given time that is smaller than the first given time, and
in the event that the measured temperature exceeds the third threshold value in a period from commencement of the operation sequence until the second given time has elapsed, stops the operation sequence, makes a transition to the halt sequence, and continues the halt sequence for the first given time.

4. The observation device of claim 2, wherein:
the vessel in which the specimen is arranged is a vessel of a plurality of different shapes, and
the controller changes the first threshold value in accordance with information specifying the vessel in which the specimen is arranged, among the vessels of a plurality of different shapes.

5. The observation device of claim 2, wherein:
the controller changes the second threshold value and the third threshold value in accordance with information specifying the vessel in which the specimen is arranged, among the vessels of a plurality of different shapes.

6. The observation device of claim 4, further comprising:
an interface that inputs information relating to the vessels of a plurality of different shapes, and
the controller specifies a vessel that a specimen is placed in from among the vessels of a plurality of different shapes based on information that is input using the interface.

7. The observation device of claim 6, further comprising:
an external control unit that can communicate with the observation device, and wherein
the interface receives instructions from the external control unit, and is input with information relating to the vessels of a plurality of different shapes.

8. The observation device of claim 4, wherein:
the controller specifies a vessel that a specimen is placed in, from among the vessels of a plurality of different shapes, based on image data that has been generated by the image sensor.

9. The observation device of claim 1, further comprising:
a focus control circuit that adjusts relative focus of the image sensor, and wherein
the controller, during execution of the operation sequence, changes imaging position of the image sensor using the imaging position change circuit, performs focusing by controlling relative focus of the image sensor using the focus control circuit, and executes an imaging operation using the image sensor, and during the halt sequence prohibits operation of the focus control circuit.

10. The observation device of claim 4, wherein:
the temperature sensor has a plurality of temperature sensors at different positions of the observation device, and temperature to be measured is determined by estimating temperature of the specimen based on outputs of the plurality of temperature sensors.

11. An observation method for an observation device that has an image sensor that forms images of a specimen, comprising:
changing imaging position of the image sensor,
measuring temperature inside the observation device and outputting this measurement result as measured temperature, and
when controlling imaging position of the image sensor and operation of the image sensor,
causing execution of an operating sequence that repeatedly executes change of imaging position of the image sensor and an imaging operation of the image sensor, and
transferring to a halt sequence, where the operating sequence is stopped if the measured temperature exceeds a given threshold value, and where a state in which change of imaging position of the image sensor and an imaging operation are prohibited continues for a given time.

12. The observation method of claim 11, further comprising:
inputting information on a vessel that the specimen is arranged in, and
changing the threshold value or the given time based on the information on the vessel.

13. The observation method of claim 11, wherein:
as the threshold value there is a first threshold value, and as the given time there are a first given time and a second given time, and
when controlling imaging position of the image sensor and operation of the image sensor,
in the event that the measured temperature does not exceed the first threshold value in a period from commencement of the operation sequence until the second given time has elapsed, continuing the operation sequence even after the second given time has elapsed, and
in the event that the measured temperature exceeds the first threshold value in a period from commencement of the operation sequence until the second given time has elapsed, making a transition to the halt sequence where the operation sequence is stopped, and continuing the halt sequence for the first given time.

14. The observation method of claim 11, wherein:
as the threshold value there is a first threshold value, a second threshold value and a third threshold value, and as the given time there are a first given time and a second given time, and
when controlling imaging position of the image sensor and operation of the image sensor,
in the event that the measured temperature has exceeded the second threshold value in a period from commencement of the operation sequence until the second given time has elapsed, and the measured temperature has not exceeded the third threshold value that is larger than the second threshold value and smaller than the first threshold value, the operation sequence is stopped, a transition is made to the halt sequence, and the halt sequence is continued for a third given time that is smaller than the first given time, and
in the event that the measured temperature exceeds the third threshold value in a period from commencement of the operation sequence until the second given time has elapsed, the operation sequence is stopped, a transition is made to the halt sequence, and the halt sequence continues for the first given time.

15. The observation method of claim 13, wherein:
the vessel in which the specimen is arranged is a vessel of a plurality of different shapes, and
when controlling imaging position of the image sensor and operation of the image sensor, the first threshold value and the second threshold value are changed in accordance with information specifying a vessel in which the specimen is arranged, from among the vessels of a plurality of different shapes.

16. The observation method of claim 13, wherein:
when controlling imaging position of the image sensor and operation of the image sensor, at least one of the second threshold value and the third threshold value are changed in accordance with information specifying a vessel in which the specimen is arranged, from among the vessels of a plurality of different shapes.

17. The observation method of claim 15, further comprising:
inputting information relating to the vessels of a plurality of different shapes, and
when controlling imaging position of the image sensor and operation of the image sensor, specifying a vessel in which the specimen is arranged, from among the vessels of a plurality of different shapes, based on information that has been input.

18. The observation method of claim 15, further comprising:
performing communication between the observation device and an external control unit, and
receiving instructions from the external control unit, and inputting information relating to the vessels of a plurality of different shapes.

19. The observation method of claim 15, further comprising:
when controlling imaging position of the image sensor and operation of the image sensor, specifying a vessel in which the specimen is arranged, from among the vessels of a plurality of different shapes, based on image data that has been generated by the image sensor.

20. A storage medium, provided within a computer of an observation device that has an image sensor for forming images of a sample, the storage medium storing a program for executing an observation method, the observation method comprising:
changing imaging position of the image sensor,
measuring temperature inside the observation device and outputting this measurement result as measured temperature, and when controlling imaging position of the image sensor and operation of the image sensor,
causing execution of an operating sequence that repeatedly executes change of imaging position of the image sensor and an imaging operation of the image sensor, and
transferring to a halt sequence, where the operating sequence is stopped if the measured temperature exceeds a given threshold value, and where a state in which change of imaging position of the image sensor and an imaging operation are prohibited continues for a given time.
